(19) United States
(12) Patent Application Publication
(10) Pub. No.: US 2019/0298669 A1
(43) Pub. Date: Oct. 3, 2019

(54) CI-1040 FOR THE TREATMENT OF VIRAL DISEASES

(71) Applicant: Atriva Therapeutics GmbH, Tubingen (DE)

(72) Inventor: Oliver Planz, Dettingen an der Erms (DE)

(21) Appl. No.: 16/368,560

(22) Filed: Mar. 28, 2019

Related U.S. Application Data
(60) Provisional application No. 62/649,859, filed on Mar. 29, 2018.

(51) Int. Cl. A61K 31/166; A61P 31/16

(52) U.S. Cl. CPC A61K 31/166; A61P 31/16

(57) ABSTRACT

The present invention relates to a method of treating viral infections comprising the administration of the MEK inhibitor CI-1040 or a pharmaceutically acceptable salt or derivative thereof. In one embodiment, the treatment of Influenza viruses in human patients is intended.

### CROSS-REFERENCE TO RELATED APPLICATION

This application claims priority under 35 U.S.C. § 119(e) from U.S. Provisional Patent Application having Ser. No. 62/649,859, filed on Mar. 29, 2018, and titled CI-1040 FOR THE TREATMENT OF VIRAL DISEASES, wherein the entirety of said provisional patent application is incorporated herein by reference.

### FIELD OF THE INVENTION

The present invention relates to a method of treating viral infections comprising the administration of the MEK inhibitor CI-1040 or a pharmaceutically acceptable salt or derivative thereof to a subject.

### BACKGROUND OF THE INVENTION

Infections with RNA or DNA viruses are a significant threat for the health of man and animal. For instance, infections with influenza viruses do still belong to the big epidemics of mankind and cause year for year a big number of casualties. In terms of the national economies, they are an immense cost factor, for instance due to unfitness for work. Infections with the Borna disease virus (BDV), which mainly affects horses and sheep, but which has also been isolated for humans and is connected to neurological diseases, equally have an enormous economic importance.

The problem of controlling in particular RNA viruses is the adaptability of the viruses caused by a high fault rate of the viral polymerases, which makes the production of suitable vaccines as well as the development of antiviral substances very difficult. Furthermore it has been found that the application of antiviral substances immediately directed against the functions of the virus, shows a good antiviral effect at the beginning of the treatment, but will quickly lead to the selection of resistant variants based on mutation. An example is the anti-influenza agent amantadine and its derivatives directed against a transmembrane protein of the virus. Within a short time after the application, resistant variants of the virus are generated. Other examples are the new therapeutics for influenza infections inhibiting the influenza-viral surface protein neuraminidase. To these belong for instance Relenza. In patients, Relenza-resistant variants have already been found (Gubareva et al., 1998). Hopes placed in this therapeutical could therefore not be fulfilled.

Because of the very small genome and thus limited coding capacity for functions being necessary for the replication, all viruses are dependent to a high degree from functions of their host cells. By exertion of influence on such cellular functions being necessary for the viral replication, it is possible to negatively affect the virus replication in the infected cell. Herein, there is no possibility for the virus to replace the lacking cellular function by adaptation, in particular by mutations, in order to thus escape from the selection pressure. This could already be shown for the influenza A virus with relatively unspecific inhibitors against cellular kinases and methyl transferases (Scholtissek and Miiller, 1991).

It is known in the art that cells have a multitude of signal transmission paths, by means of which signals acting on the cells are transmitted into the cell nucleus. Thereby the cell is capable to react to external stimuli and to react by cell proliferation, cell activation, differentiation, or controlled cell death. It is common to these signal transmission paths that they contain at least one kinase activating by phosphorylation at least one protein subsequently transmitting a signal. When observing the cellular processes induced after virus infections, it is found that a multitude of DNA and RNA viruses preferably activate in the infected host cell a defined signal transmission path, the so-called Raf/MEK/ERK kinase signal transmission path (Benn et al., 1996; Bruder and Kovesdi, 1997; Popik and Pitha, 1998; Rodems and Spector, 1998). This signal transmission path is one of the most important signal transmission paths in a cell and plays a significant role in proliferation and differentiation processes. Growth factor-induced signals are transmitted by successive phosphorylation from the serine/threonine kinase Raf to the dual-specific kinase MEK (MAP kinase kinase/ERK kinase) and finally to the kinase ERK (extracellular signal regulated kinase). Whereas as a kinase substrate for Raf, only MEK is known, and the ERK isoforms were identified as the only substrates for MEK, ERK is able to phosphorylate a whole number of substrates. To these belong for instance transcription factors, whereby the cellular gene expression is directly influenced (Cohen, 1997; Robinson and Cobb 1997; Treisman, 1996).

The drawback of prior art antiviral active substances is that they are either directed against a viral component and thus quickly lead to resistances (cf. amantadine), or act in a too broad and unspecific manner against cellular factors (for example methyl transferase inhibitors), and significant side effects are to be expected. Consequently, none of the substances being active against cellular factors is known to have been developed to a therapeutical for virus diseases. On the other hand, the inhibition of other kinases, for instance the inhibition of the kinase JNK of the MEKK/SEK/JNK signal transmission path, can increase the virus multiplication. Further it is known that the increased activation of again other kinases, for instance of the protein kinase C (PKC), inhibits the replication of viruses (Driedger and Quick, WO 92/02484).

With regard to the cellular processes induced after a virus infection, it is found that a multitude of DNA and RNA viruses activate, in the infected host cell, a defined signal transduction pathway, the so-called Raf/MEK/ERK kinase cascade.

This kinase cascade belongs to the most important signaling pathways in the cell and plays an essential role in proliferation and differentiation processes.

Growth-factor induced signals are transferred by successive phosphorylation from the serine/threonine kinase Raf to the dual specific kinase MEK (MAP kinase kinase/ERK kinase) and finally to the kinase ERK (extracellular signal regulated kinase). Whilst as a kinase substrate of Raf, only MEK is known, and the ERK isoforms have been identified for MEK as the only substrate, ERK can phosphorylate quite a number of substrates. Hereto belong for instance the phosphorylation of transcription factors, which leads to a direct modification of the cellular gene expression.

The investigation of this signaling pathway in cellular decision processes has led to the identification of several pharmacological inhibitors, which inhibit the signaling pathway, among other positions, on the level of MEK, i.e. at the ‘bottleneck’ of the cascade.

The MEK inhibitors CI-1040, PD0325901, AZD6244, GDC-0973, RDEA119, GSK1120212, AZD8330, RO5126766, RO4987655, TAK-733 and AS703026 are known in the art and, for example, shown in FIG. 4 of Fremin and Meloche (2010).

Neuraminidase (also known as sialidase, acylneuraminyl hydrolase, and EC 3.2.1.18) is an enzyme common among animals and a number of microorganisms. It is a glycohydrolase that cleaves terminal alpha-ketosidically linked sialic acids from glycoproteins, glycolipids and oligosaccharides. Many of the microorganisms containing neuraminidase are pathogenic to man and other animals including fowl, horses, swine and seals. These pathogenic organisms include influenza virus.

Neuraminidase has been implicated in the pathogenicity of influenza virus. It is thought to help the elution of newly synthesized virons from infected cells and assist in the movement of the virus (through its hydrolase activity) through the mucus of the respiratory tract.

A class of specific anti-influenza agents, the neuraminidase inhibitors, has demonstrated inhibition of both influenza A and B viruses. Oseltamivir is used for the treatment of viral infections; however, it does not treat nasal congestion. Oseltamivir is the ethyl ester prodrug of the carbocyclic transition state sialic acid analog RO 64-0802 (GS4071), a potent and selective inhibitor of influenza A and B virus neuraminidases. Oral oseltamivir has been approved for treatment of acute influenza in the United States in 1999. It has demonstrated efficacy both in treating and preventing influenza illness.

Oseltamivir phosphate is a prodrug of oseltamivir carboxylate (oseltamivir), an inhibitor of the neuraminidase glycoprotein essential for replication of influenza A and B viruses. Oseltamivir is available from Roche Pharma™ AG (Switzerland). Alternatively, oseltamivir can be prepared according to the methods described in U.S. Pat. No. 5,763,483 to Bischofberger et al and U.S. Pat. No. 5,866,601 to Lew et al. About 10-15% of patients taking oseltamivir experience nausea and vomiting. Patients with kidney dysfunction should take lower doses.

Zanamivir (Relenza) is an orally inhaled powder currently approved in 19 countries for the treatment of, and in two for the prophylaxis of influenza A and B. Zanamivir is a competitive inhibitor of the neuraminidase glycoprotein, which is essential in the infective cycle of influenza viruses. It closely mimics sialic acid, the natural substrate of the neuraminidase. Over the last few years, a number of events have resulted in changes to the zanamivir prescribing information which now contains warnings of bronchospasm, dyspnea, rash, urticaria and allergic type reactions, including facial and oropharyngeal oedema.

Peramivir is a neuraminidase inhibitor, acting as a transition-state analogue inhibitor of influenza neuraminidase and thereby preventing new viruses from emerging from infected cells.

It is known that neuraminidase inhibitors are not effective for all influenza viruses and a resistance can be developed by new generation of influenza virus strain.

As viruses and influenza virus in particular very often develop resistances against antiviral treatment, there is a need for new and improved therapy options.

CI-1040 is a clinically tested MEK inhibitor. The drug CI-1040, also known as 2-(2-chloro-4-iodophenylamino)-N-(cyclopropylmethoxy)-3,4-difluorobenzamide, is an ATP non-competitive MEK1/2 inhibitor and was originally developed as an anti-tumor drug where it showed low toxicity (Barrett et al., 2008; Lorusso et al., 2005). It directly inhibits MEK1 with a 50% inhibitory concentration (IC50) of 17 nM. The structure of CI-1040 is shown in formula (I):

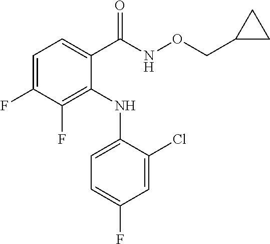 [SMILES: O=C(NOCC1CC1)c1ccc(F)c(F)c1Nc1ccc(F)cc1Cl]

Influenza is an acute respiratory disease caused by infection with Influenza viruses (IV). The disease affects the upper and/or lower respiratory tract and is often accompanied by systemic signs and symptoms such as fever, headache, myalgia, and weakness. Outbreaks of disease of variable extent and severity occur nearly every winter. They result in significant morbidity in the general population and increased mortality rates among certain high-risk patients mainly as a result of pulmonary complications.

The H1N1 pandemic in 2009 clearly demonstrates that Influenza A virus (IAV) has a strong impact on global health systems (Mackey and Liang, 2012; Monto et al., 2011; Robertson and Inglis, 2011). Besides preventive vaccination, only a few antiviral drugs are approved. This highlights the urgent need for additional effective antivirals to better control the infection. Notably, in the early phase of a pandemic, when no vaccine is available, antivirals are the stand-alone treatment. Moreover, the appearances of IAV that are resistant against currently approved antivirals underline the urgent need for new and amply available antiviral drugs (Moss et al., 2010).

It is of great importance that antiviral therapy of viral infections is started as soon as possible after infection. E.g., the time window to start an influenza therapy using oseltamivir is only up to 24 h. Very often, a patient visits a physician after this period of time rendering the treatment with standard anti-viral treatments futile. Further, antiviral treatments may be contraindicated because of possible drug interactions, especially in elder people, who often have to take one or more medications continuously. Very often, it is not easy to step the taking of such medication. As a consequence, the time frame for starting an antiviral therapy already is passed before these patients are suited for antiviral therapy.

In view of the prior art, it is clear that there is the need of compounds and compositions effective in the treatment of virus diseases in particular in diseases caused by influenza virus, especially for late start of treatment. In addition, there is a need for treatment of virus infections, wherein the virus is resistant to antiviral therapy.

### SUMMARY OF THE INVENTION

This need is obviated by using the MEK inhibitor CI-1040 or a pharmaceutically acceptable salt or derivative thereof for treating a viral infection.

The present invention relates to a method for treating a subject having a viral infection, comprising: administering to the subject the MEK inhibitor CI-1040 or a pharmaceutically acceptable salt or derivate thereof, wherein administering treats a viral infection in the subject.

Preferably, the subject has been symptomatic for a viral infection for at least 24 h, at least 36 h or at least 48 h.

Preferably, the treating is started at least 24 hours and 48 hours post onset of disease.

Preferably, the viral infection is caused by a negative RNA strand virus. Preferably, the virus is influenza virus, more preferably the virus is influenza A virus (IAV) or influenza B virus (IBV), even more preferably, the influenza A virus is selected from the group consisting according to H1N1, H2N2, H3N2, H5N6, H5N8, H6N1, H7N2, H7N7, H7N9, H9N2, H10N7, N10N8 or H5N1 or the Influenza B virus is selected from the group consisting of IBV type Yamagata and Victoria.

Preferably, the virus is resistant to antiviral treatment, wherein more preferably the antiviral treatment is administration of oseltamivir, zanamivir, peramivir, amantadine, rimantadine, favipiravir, baloxavir marboxil or pimodivir.

Preferably, the virus resistant to antiviral treatment is a H1N1 H275Y mutant.

Preferably, the subject is human.

### FIGURES

FIG. 1A shows the dose-dependent inhibition of A/Regensburg/06/2009 by CI-1040 (left panel), the determination of the EC₅₀ for Cl-1040 (middle panel) and of the CC₅₀ (right panel).

FIG. 1B shows a broad antiviral range of CI-1040 at 10 μM against different influenza A viruses.

FIG. 1C shows that Cl-1040 can inhibit a Tamiflu® (oseltamivir)-resistant IAV strain.

FIG. 2 shows immunofluorescence staining of human lung epithelial cells. Constituents of the cell nuclei with DAPI (first column) and of the viral RNP complexes, PB1 (second column) and NP (third column) were stained. In the fourth column the images were merged. The Figure shows that CI-1040 blocks the export step of influenza A, H1N1, in human lung epithelial cells (A549).

FIG. 3A shows the efficacy of CI-1040 in the treatment of C57BL/6 mice infected with influenza virus H1N1pdm09 and a timeline of the treatments. Results are presented as virus titer (log 10) pfu/ml (left lower panel) or % virus titer relative to control, whereas control was set to 100% (right lower panel). The experiment was performed twice independently.

FIG. 3B shows the efficacy of CI-1040 in the treatment of C57BL/6 mice infected with influenza virus H1N1pdm09 and proves the prolonged treatment window in comparison to oseltamivir (Tamiflu®).

FIG. 4 shows in vitro studies with the MEK-Inhibitor CI-1040 against H5N1 influenza virus.

FIG. 5 shows results of the Oral treatment of influenza virus infected mice with CI-1040 or PD-03250901.

### DETAILED DESCRIPTION

Influenza viruses (IV) continue to pose an imminent threat to human welfare. Yearly re-occurring seasonal epidemic outbreaks and pandemics with high mortality can occur. Besides vaccination against a limited number of viral strains only a few antiviral drugs are available, which are losing their effectiveness as more and more IV strains become resistant. Thus, new antiviral approaches that omit IV resistance are urgently needed. Here, the dependency on the cellular Raf/MEK/ERK signaling pathway for IV replication opens a new perspective. In consequence, the inventors studied the antiviral potential of the MEK inhibitor Cl-1040 (PD184352) and show that Cl-1040 surprisingly and significantly reduces virus titers in vitro via retention of viral RNP complexes in the cell nucleus. Furthermore, Cl-1040 is surprisingly effective against a broad range of IV strains, including highly pathogenic avian IV, as well as against a Tamiflu®-resistant IV strain. Using a mouse model, the inventors demonstrate that Cl-1040 can reduce IV lung titers in vivo. Surprisingly, the treatment window for Cl-1040 expands to at least 48 h post infection when Tamiflu® treatment has no effect. In conclusion, Cl-1040 offers an interesting perspective for anti-IV approaches.

All viruses depend on cellular factors and mechanisms for their replication and so do Influenza Viruses (IV). They acquired the ability to highjack cellular factors for its own purpose (Ludwig et al., 2003). Given these dependencies, cellular virus-supportive functions are promising candidates for novel antiviral intervention (Ludwig, 2011; Ludwig et al., 2003; Planz, 2013). Besides directly targeting virus as shown for the approved neuraminidase-inhibitors, such as Tamiflu® or the M2-ionchannel blockers like amantadine, a new and promising antiviral strategy to fight influenza is based on the fact that IV, as intracellular pathogen, is strongly dependent on the cellular signaling machinery (Gong et al., 2009; Ludwig, 2009).

IV must pass cellular barriers during their intracellular replication. As such the viral ribonucleoproteins (RNPs), comprising the genome segments, are transported from the cytoplasm to the nucleus, where viral genome replication takes place, and back to the cell membrane later in the replication cycle, when progeny virus particles are released from the infected cell. Remarkably, the nuclear RNP export was shown to be strongly dependent on the virus-induced Raf/MEK/ERK signal transduction pathway (Pleschka et al., 2001; Ludwig et al., 2004). Hence, pathways that are required for the virus to cross intracellular barriers, such as the nuclear membrane, are most favorable for antiviral intervention.

A potential advantage of antiviral strategies that target intracellular signaling pathways is their reduced likeliness to induce viral resistance in comparison to those that directly target viral replication. This has already been shown for several compounds (Ludwig et al., 2004; Mazur et al., 2007). In contrast, potential adverse effects of inhibitors of intracellular signaling pathways must also be taken into consideration, since they interfere with the host cell machinery and with substantial cellular functions.

As described above, the MEK inhibitor of the invention is CI-1040, as well as pharmaceutically acceptable salts and may also include derivatives and metabolites thereof. A derivative, also known as structural analog, is a compound having a structure similar to that of another compound, but differing from it in respect to a certain component. It can differ in one or more atoms, functional groups, or substructures, which are replaced with other atoms, groups, or substructures.

Surprisingly it has been found that the administration of CI-1040 alone is effective in the treatment of viral infections as shown in the Examples 1, 2 and 4. CI-1040 is not directed against the functions of the virus but selectively inhibits MEK, a cellular enzyme and via this selective effect inhibits the viral replication of virus. Hence, CI-1040 is also effective against viruses, which are resistant to standard antiviral treatment, which targets the virus itself. As mentioned above, for influenza, such treatments are neuraminidase inhibitors such as oseltamivir (Tamiflu®) or zanamivir or M2 inhibitors such as amantadine or rimantadine, etc. Further antiviral treatments include the administration of peramivir, favipiravir, baloxavir marboxil and/or pimodivir.

The present invention relates to the treatment of a subject having a viral infection, comprising administering to the subject the MEK inhibitor CI-1040 or a pharmaceutically acceptable salt or derivative thereof, wherein administering treats a viral infection in the subject. In addition, CI-1040 can be administered to patients after treatment with standard antivirals has failed.

As shown in Example 1, CI-1040 can suppress replication of H1N1pdm09 influenza virus (A/Regensburg/D6/2009 (H1N1)) in vitro in human alveolar epithelia cells (A549) at non-toxic concentrations (CC50=>312.3 μM). The concentration at which viral propagation is reduced by 50% (effective concentration; EC50) was 0.026 μM, which is in the same range as oseltamivir for this virus (data not shown). The selectivity index is 12.012. EC50 value of CI-1040 to inhibit 50% of tumor cell growth is ranging from 0.12 to 0.18 M (Sebolt-Leopold et al., 1999). Thus, in cell culture an approximately 10-fold lower concentration of CI-1040 compared to anti-tumor activity is needed for antiviral activity. This may be explained by the fact that in tumor cells the Raf/MEK/ERK pathway is permanently activated due to mutations. In influenza virus infected cells the pathway is only activated for a short time (Pleschka et al. 2001). Thus, Example 1 shows that CI-1040 can inhibit an influenza virus at non-toxic concentrations in a comparable manner to oseltamivir.

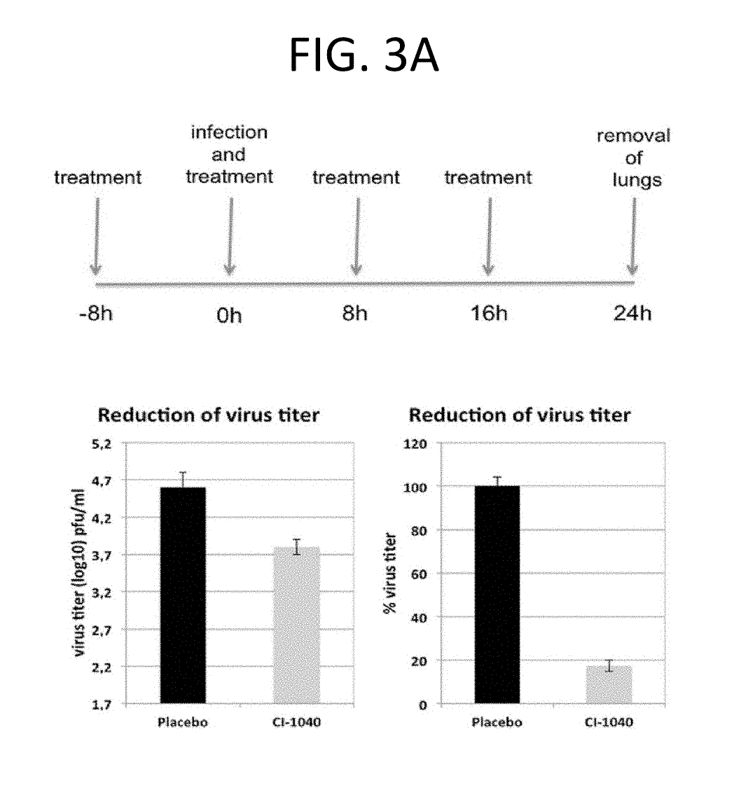

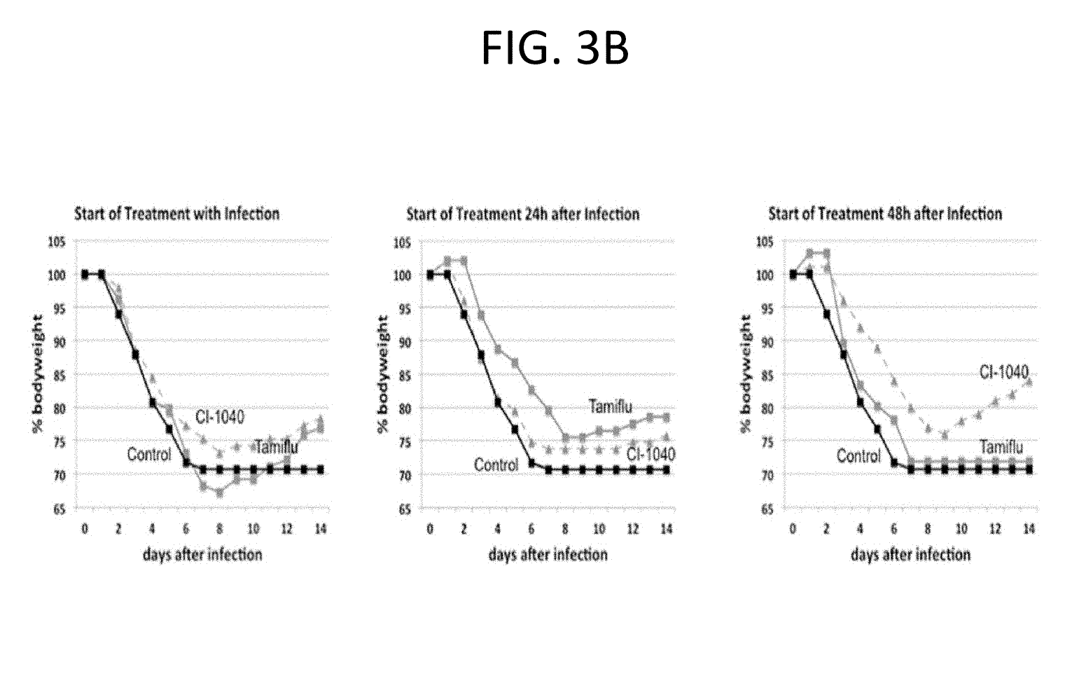

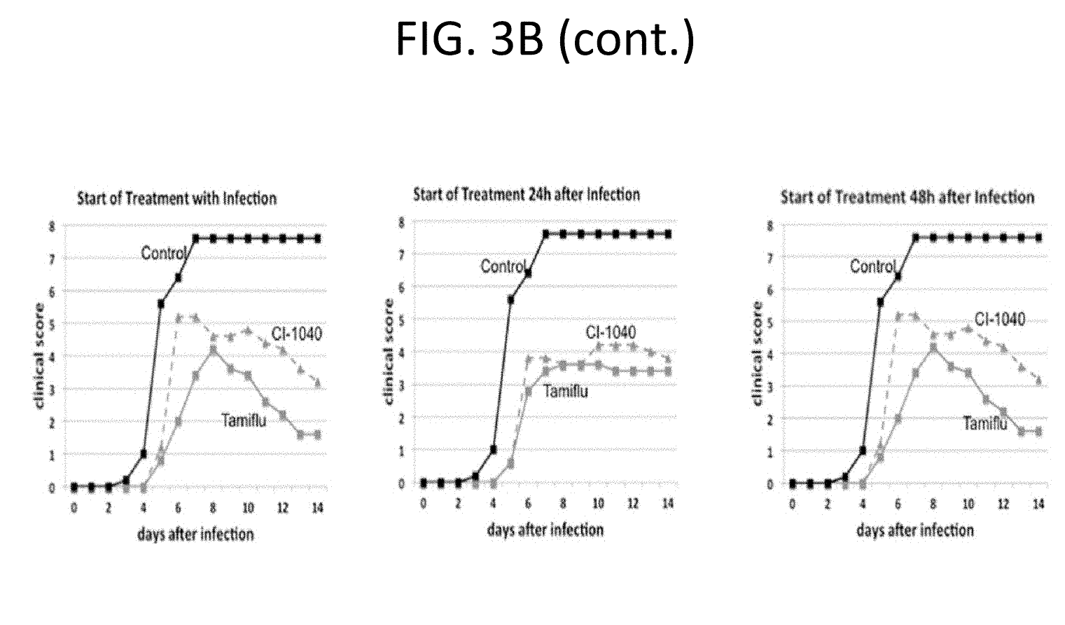

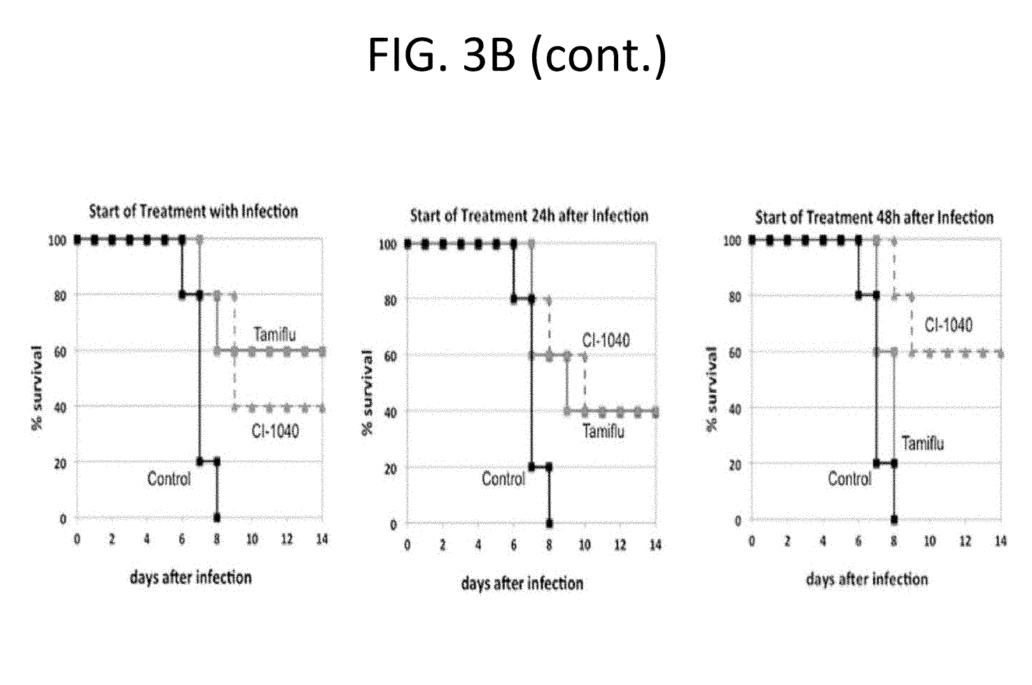

This is confirmed in Example 3, where it was shown that CI-1040 possesses anti-influenza virus activity also in a mouse model. CI-1040 treatment via the per os route of H1N1pdm09-infected mice (5×MLD50) resulted in more than 80% reduction of the amount of virus in the lung (FIG. 3A) at a time-point where the viral load in placebo-treated mice is high (24 hours post infection). This indicates that the metabolism of the compound is sufficient to reach the lung and that the antiviral activity found in vitro is also evident during infection of an organism. Example 4 supports this data.

Surprisingly, it has been found that CI-1040 is also effective if the treatment is started at 48 h after infection. By comparison, as shown in Example 3, oseltamivir is only effective in the treatment of influenza A in mice, if the treatment is started up to 24 h post infection. By using CI-1040, the inventors surprisingly found that the treatment window can be prolonged to at least 48 h, most likely more. This property of CI-1040 could not be expected. Hence, CI-1040 is especially useful in the treatment of viral diseases such as influenza, if a proper diagnosis could not be made timely, so that a standard viral therapy would be without effect or in cases where the standard antiviral treatment was found to be ineffective.

In Example 3, the inventors investigated the “treatment window”—the time of the treatment start after infection. When treatment started with the infection (left panel of FIG. 3B), the drop of bodyweight was comparable in all groups. All animals developed disease and all control mice (5/5) died. In the Tamiflu® group 2/5 (40%) mice died whereas 3/5 mice, treated with CI-1040, died (60%). In particular, the fact that Tamiflu®-treated mice died was not expected since it was published that Tamiflu® protects all mice when treatment started with the infection (Baranovich et al., 2014; Yen et al., 2005). Similar results were observed, when treatment started 24 h after infection (FIG. 3B, middle panel). When treatment started 48 h after infection, however, (FIG. 3B, right panel), all Tamiflu®-treated mice died (5/5, 100%). This failure of protection if administered more than 48 hours post infection was already described for highly pathogenic strains (Leneva et al., 2000). However, it was surprisingly found that if the start of treatment was 48 h after the infection only 2/5 (40%) CI-1040-treated mice died. Also, a benefit in bodyweight, clinical score and overall survival was still found in CI-1040-treated mice. This antiviral effect was found using a concentration of CI-1040 that was below the concentrations used in anti-tumor mouse models, indicating that a lower amount of CI-1040 is required to achieve an antiviral effect compared to an anti-tumor effect.

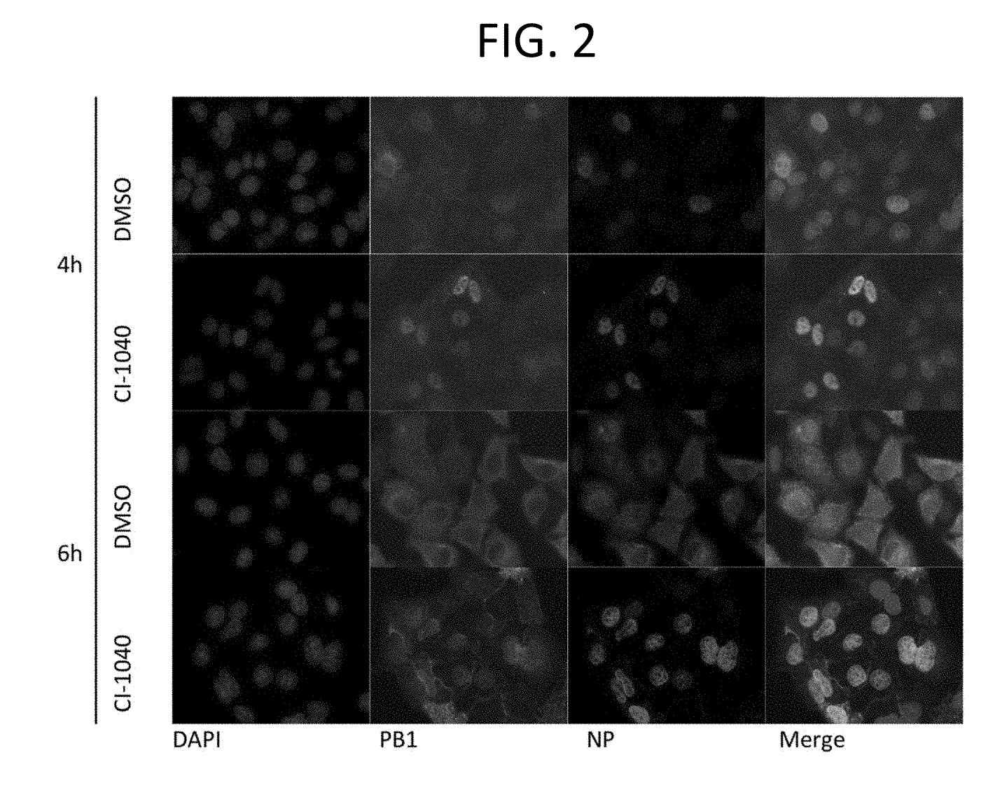

According to the FDA label, oseltamivir (Tamiflu®) is currently indicated for the treatment of influenza in patients who have been symptomatic for no more than 2 days. This corresponds to what was observed in the mouse model in Example 3, where oseltamivir is no longer effective if it is administered more than 48 h post infection with influenza virus. In contrast, CI-1040 was effective even 48 h post infection. Hence, it has been shown that CI-1040 has a longer treatment window than oseltamivir. For this reason, it is likely that the effects observed in the mouse model can be extrapolated to human patients, meaning that CI-1040 or a pharmaceutically acceptable salt or a derivative thereof can be administered to patients more than 2 days after onset of symptoms of a viral infection. This also makes sense when the method of action of CI-1040 is compared to that of oseltamivir. As mentioned above, oseltamivir is thought to block the elution of virions from the infected cells. For this reason, it is only effective if the virions have not yet left the cell. It was found that CI-1040, by contrast, blocks the MEK pathway that is necessary for viral replication, so that the viral RNP complexes are retained in the cell nucleus. Regarding the molecular mode of action of MEK inhibition, the inventors have previously shown that inhibition of the kinase with the prototype inhibitor U0126 leads to a blockade of nuclear cytoplasmic transport of viral RNP complexes (Pleschka et al., 2001). This could be confirmed for CI-1040 (see also Example 2). While, RNPs are present in the nucleus at 4 h post infection in control and CI-1040 treated cells (FIG. 2, upper two lanes), they translocate to the cytoplasm at 6 hours post infection to be packaged into new virions in control cells (FIG. 2, third lane). This export step is readily blocked in the presence of CI-1040 as evidenced by predominant nuclear staining of the viral NP, the major constituent of the RNPs, as well as the PB1 polymerase, which is associated with RNPs (FIG. 2, lower lane).

In this context and in light of the data in Examples 2 and 3, discussed above, it has been shown that treatment window for CI-1040 in human patients is larger than for oseltamivir.

Accordingly, the method of the invention also relates to treating a viral infection in subjects that have been symptomatic for at least 24 hours, at least 36 hours, or at least 48 hours. These times of course include all time frames in between, such as 25, 26, 27, 28, 29, 30, 31, 32, 33, 34, 35, 37, 38, 39, 40, 41, 42, 43, 44, 45, 46 or 47 hours for the subject being symptomatic. In a preferred embodiment, the subject has been symptomatic for at least 48 hours before treatment. For example, treatment can be started between 24 and 48 hours after onset of disease. In this context it should be noted that “being symptomatic” and “onset of disease” can be used interchangeably. However, the treatment could also be started at least 60 h, at least 72 h or at least 96 h post infection. In cases where CI-1040 or a pharmaceutically acceptable salt or derivative thereof is administered when a different antiviral was already found to be ineffective, CI-1040 or a pharmaceutically acceptable salt or derivative thereof can be administered after 24 hours, when the treatment with the previous antiviral has been completed.

The expression “a patient or subject who is symptomatic” for a viral infection is defines as a patient or subject showing symptoms of a viral infection. In case of an influenza virus infection, the symptoms include fever, cough, nasal congestion, runny nose, sneezing, sore throat, fatigue, headache or muscle aches. Not all of these symptoms may occur after an influenza virus infection. Typically, influenza starts suddenly chills or a chilly sensation, which accompanies the sudden onset of (high) fever. This point in time may be defined as the beginning of the period of being symptomatic.

As already described above, the MEK inhibitor of the invention is CI-1040, as well as its pharmaceutically acceptable salts, derivatives and metabolites thereof.

In one aspect, the method of the invention is for the treatment of a viral disease which is an infection caused by negative RNA strand virus. More preferably, the viral disease is caused by an influenza virus; even more preferably is caused by influenza A or B viruses. Influenza A viruses are for example H1N1, H2N2, H3N2, H5N6, H5N8, H6N1, H7N2, H7N7, H7N9, H9N2, H10N7, N10N8 or H5N1. Influenza B viruses are for example the Yamagata or Victoria type.

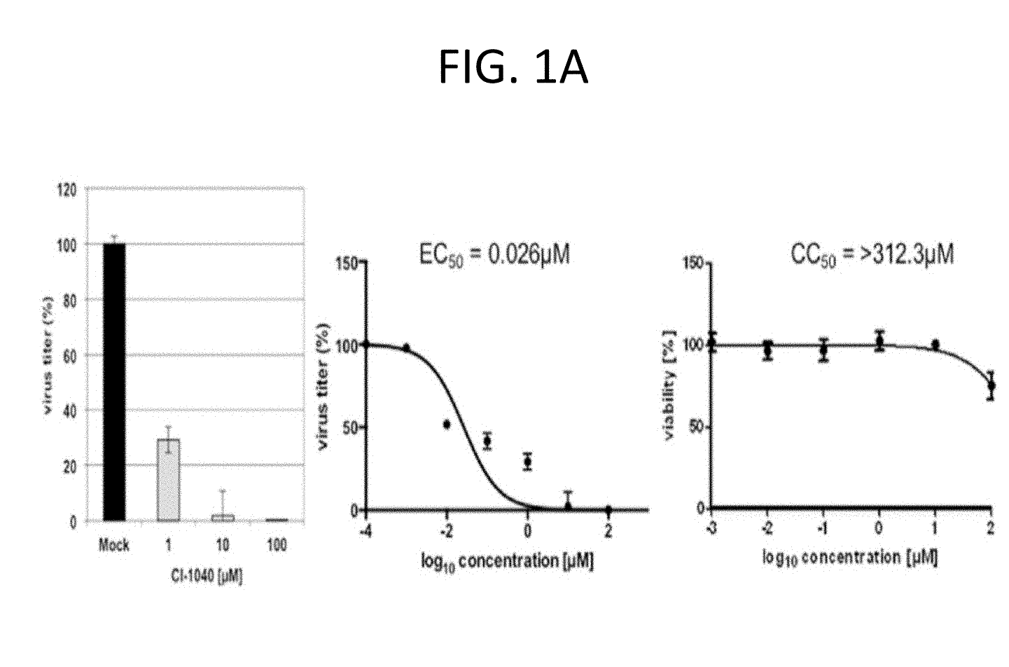

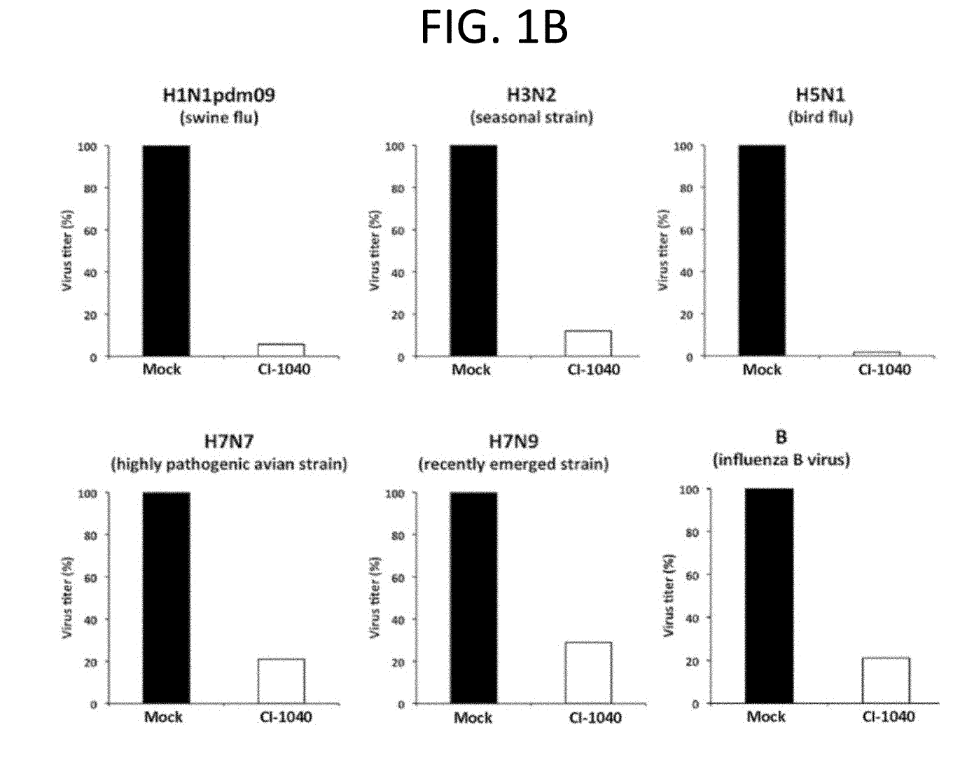

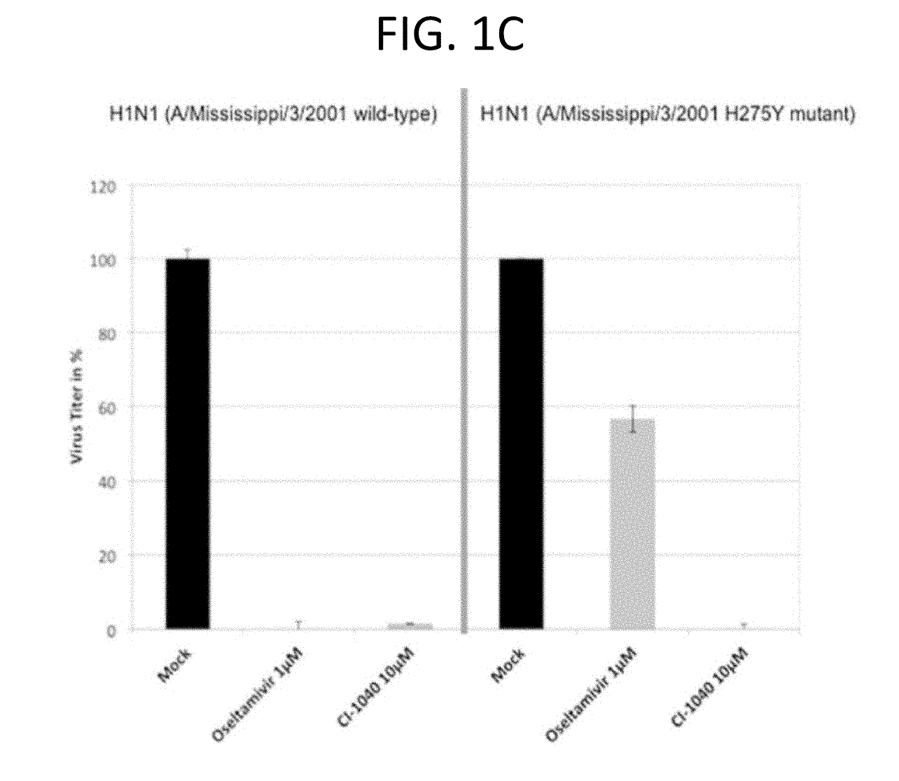

Besides the H1N1pdm09 subtype the H3N2 subtype is circulating among the human population and contributes to the annual epidemics (Broberg et al., 2015). This strain originally caused the 1968 pandemic outbreak or the so-called “Hong Kong-Flu” and has then adapted to cause “only” seasonal epidemics. There are occasional infections of humans with “bird flu” viruses of the subtypes H5 and H7 displaying a very high mortality (Morens et al., 2013; Osterholm et al., 2012). H5N1 viruses first appeared in humans in 1997 and since then have caused numerous infections of humans. Furthermore, during an outbreak of an H7N7 virus of avian origin in the Netherlands in 2003 several people were infected and one person died (Fouchier et al., 2004). In 2013, a novel “bird flu” virus of the H7N9 subtype emerged in China that already killed more than 400 (428 of 1230 confirmed cases as of Feb. 22, 2017) people (Ref.: FAO). Moreover, besides IAV also influenza B virus is found to cause epidemics. Thus, the activity of 10 M CI-1040 against prototype strains of these aggressive virus subtypes was tested and results are shown in FIG. 1B (see also Example 1). A consistently strong antiviral activity could be demonstrated. Cl-1040 can inhibit several IAV strains, indicating a broad activity of Cl-1040 against human IAV and influenza B virus causing seasonal epidemics as well as against avian IAV that represent an imminent threat to the human population as they have the potential to become pandemic.

The emergence of viral resistance to licensed influenza drugs is a rising issue. High frequency of resistance to the M2-ion channel blocker amantadine in clinical isolates in the US have led to the conclusion that M2 inhibitors should not be used for the treatment and prophylaxis of influenza until susceptibility to these drugs has been reestablished among circulating influenza A isolates (Bright et al., 2006). In contrast, it was reported that the resistance to neuraminidase inhibitors (Tamiflu® and Relenza®) is generally low. During clinical trials of Tamiflu® in seasonal influenza only a low percentage of resistance has been reported (Aoki et al., 2007). However, more worrying rates of resistance have been first detected in a smaller study in Japanese children where 18% of all isolates were resistant (Kiso et al., 2004). Since then, the number of reports on viral resistance to Tamiflu® has rapidly increased, including findings on the emergence of influenza B viruses as well as A/H5N1, A/H7N9 and pandemic A/H1N1 type viruses that are insensitive against the compound. During the 2007-2008 influenza season Tamiflu®-resistant variants of seasonal influenza H1N1 emerged, leading to a global subtype wide resistance in the following years (Lackenby et al., 2008; Thorlund et al., 2011). Besides the problem of resistance, recent studies also questioned the clinical benefit of Tamiflu® based on a critical evaluation of clinical trial data (Jefferson et al., 2009; Jefferson et al., 2014).

With the present MEK inhibitor CI-1040 which, as has been shown herein, targets an intracellular protein, unresponsiveness of the drug against influenza virus might be excluded. Mutations in viral proteins cannot be excluded, but to escape the influence of MEK-inhibitor treatment the virus need to change its dependency on the Raf/MEK/ERK signaling pathway for nuclear export of its RNP-complexes. This would result in a change of the biology of the virus. In the present invention, it could be demonstrated that CI-1040 is effective against a neuraminidase inhibitor (oseltamivir)-resistant IAV strain A/Mississippi/3/2001 (H1N1). The inventors took advantage of the fact that this virus exists as an oseltamivir-resistant H1N1 variant, which differs from the wild type only in the H275Y resistance mutation in the neuraminidase. As shown in FIG. 1C oseltamivir and CI-1040 are very potent to inhibit the growth of the A/Mississippi/3/2001 wild type strain (left part of FIG. 1C). No virus was detectable after oseltamivir treatment and only 1.5% of the virus was found after CI-1040 treatment. In contrast, when the antiviral effect was investigated using the mutant strain with the H275Y mutation (right part of FIG. 1C) oseltamivir had lost most of its effectiveness (only 43% reduction), while CI-1040 showed a comparable antiviral effect as against the wild type strain (99.9% reduction).

As further shown in Example 1, CI-1040 is also effective against influenza viruses, which are resistant to antiviral treatment. This is a feature of CI-1040 that solves a problem of many standard antiviral treatments and allows CI-1040 to be administered after treatment with standard antivirals has failed. Standard antiviral treatment as used herein is defined as a treatment with a drug that has been approved for use as an antiviral and which is effective in inhibiting the development of the viral pathogen at any step of its life cycle. Examples include entry inhibitors, uncoating inhibitors, inhibitors of reverse transcription, polymerase inhibitors, endonuclease inhibitors, protein maturation inhibitors, integrase inhibitors, transcription inhibitors, translation inhibitors, protease inhibitors, virion assembly inhibitors or virion release inhibitors. In the context of influenza viruses, there are two different standard approaches of antiviral treatment known: neuraminidase inhibitors and M2 protein inhibitors.

Neuraminidase inhibitors (NAIs) are a class of drugs which block the neuraminidase enzyme. They are commonly used as antiviral drugs because they block the function of viral neuraminidases of the influenza virus, by preventing its reproduction by budding from the host cell. Oseltamivir (Tamiflu) a prodrug, Zanamivir (Relenza), Laninamivir (Inavir), and Peramivir belong to this class. Unlike the M2 inhibitors, which work only against the influenza A, neuraminidase inhibitors act against both influenza A and influenza B. The neuraminidase inhibitors oseltamivir and zanamivir were approved in the US and Europe for treatment and prevention of influenza A and B.

The antiviral drugs amantadine and rimantadine inhibit a viral ion channel (M2 protein), thus inhibiting replication of the influenza A virus. These drugs are sometimes effective against influenza A if given early in the infection but are ineffective against influenza B viruses, which lack the M2 drug target. Influenza viruses readily develop resistances against M2 inhibitors.

The antiviral drug favipiravir is an inhibitor of RNA viral polymerases. The antiviral drug baloxavir marboxil is an inhibitor of the influenza virus endonuclease PA. Both drugs are licensed for influenza treatment in Japan. The antiviral drug pimodivir is a blocker of the influenza viral PB2 protein and has been shown to be efficient against uncomplicated influenza A in clinical trials. All three drugs, favipiravir, baloxavir marboxil and pimodivir have been shown to provoke drug resistant variants in experimental models or clinical testings.

Accordingly, in one embodiment the virus is resistant to antiviral treatment. As outlined herein, the antiviral treatment may relate to the administration of neuraminidase inhibitors, such as oseltamivir, zanamivir, laninamivir and peramivir, and/or administration of M2 inhibitors such as amantadine and rimantadine. Accordingly, the antiviral treatment may be the administration of oseltamivir, zanamivir, amantadine and/or rimantadine. In another embodiment, the antiviral treatment relates to the administration of oseltamivir, zanamivir, peramivir, amantadine, rimantadine, favipiravir, baloxavir marboxil and/or pimodivir. As shown in Example 1, CI-1040 is also effective against the Influenza A strain H1N1 A/Mississippi/3/2001 H275Y mutant, which is resistant to oseltamivir. Accordingly, in one embodiment, the virus is H1N1 A A/Mississippi/3/2001 H275Y mutant and in another embodiment, the virus resistant to antiviral treatment is H1N1 A/Mississippi/3/2001 H275Y mutant.

In one embodiment, the virus is H1N1 A/Mississippi/3/2001 wild type or H1N1pdm09, for which CI-1040 is also effective as shown in Example 1.

In the method of the invention, the MEK inhibitor CI-1040 may be administered orally, intravenously, intrapleurally, intramuscularly, topically or via inhalation. Preferably, the MEK inhibitor is administered via nasal inhalation or orally.

The subject or patient of the invention can be a mammal or a bird. Examples of suitable mammals include, but are not limited to, a mouse, a rat, a cow, a goat, a sheep, a pig, a dog, a cat, a horse, a guinea pig, a canine, a hamster, a mink, a seal, a whale, a camel, a chimpanzee, a rhesus monkey and a human. Examples of suitable birds include, but are not limited to, a turkey, a chicken, a goose, a duck, a teal, a mallard, a starling, a Northern pintail, a gull, a swan, a Guinea fowl or water fowl, to name a few. Human patients are a particular embodiment of the present invention.

In particular embodiments, the subject is a human subject, which optionally is more than 1 year old and less than 14 years old, between the ages of 50 and 65, between the ages of 18 or 50, or older than 65 years of age. In other embodiments the subject is a human subject, selected from the group consisting of subjects who are at least 50 years old, subjects who reside in chronic care facilities, subjects who have chronic disorders of the pulmonary or cardiovascular system, subjects who required regular medical follow-up or hospitalization during the preceding year because of chronic metabolic diseases, renal dysfunction, hemoglobinopathies, or immunosuppression.

In the method of the invention, the MEK inhibitor CI-1040 or a pharmaceutically acceptable salt or derivative thereof is administered in a therapeutically effective amount.

The therapeutically effective amount for each active compound can vary with factors including but not limited to the activity of the compound used, stability of the active compound in the patient's body, the severity of the conditions to be alleviated, the total weight of the patient treated, the route of administration, the ease of absorption, distribution, and excretion of the active compound by the body, the age and sensitivity of the patient to be treated, adverse events, and the like, as will be apparent to a skilled artisan. The amount of administration can be adjusted as the various factors change over time.

The pharmaceutical composition comprising the MEK CI-1040 inhibitor or a pharmaceutically acceptable salt or derivative thereof may be in the form of orally administrable suspensions or tablets; nasal sprays, sterile injectable preparations (intravenously, intrapleurally, intramuscularly), for example, as sterile injectable aqueous or oleaginous suspensions or suppositories. When administered orally as a suspension, these compositions are prepared according to techniques available in the art of pharmaceutical formulation and may contain microcrystalline cellulose for imparting bulk, alginic acid or sodium alginate as a suspending agent, methylcellulose as a viscosity enhancer, and sweeteners/flavoring agents known in the art. As immediate release tablets, these compositions may contain microcrystalline cellulose, dicalcium phosphate, starch, magnesium stearate and lactose and/or other excipients, binders, extenders, disintegrants, diluents, and lubricants known in the art. The injectable solutions or suspensions may be formulated according to known art, using suitable non-toxic, parenterally acceptable diluents or solvents, such as mannitol, 1,3-butanediol, water, Ringer's solution or isotonic sodium chloride solution, or suitable dispersing or wetting and suspending agents, such as sterile, bland, fixed oils, including synthetic mono- or diglycerides, and fatty acids, including oleic acid.

The pharmaceutical compounds in the method of present invention can be administered in any suitable unit dosage forms. Suitable oral formulations can be in the form of tablets, capsules, suspension, syrup, chewing gum, wafer, elixir, and the like. Pharmaceutically acceptable carriers such as binders, excipients, lubricants, and sweetening or flavoring agents can be included in the oral pharmaceutical compositions. If desired, conventional agents for modifying tastes, colors, and shapes of the special forms can also be included.

For injectable formulations, the pharmaceutical compositions can be in lyophilized powder in admixture with suitable excipients in a suitable vial or tube. Before use in the clinic, the drugs may be reconstituted by dissolving the lyophilized powder in a suitable solvent.

### Definitions

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms.

As used herein, the conjunctive term “and/or” between multiple recited elements is understood as encompassing both individual and combined options. For instance, where two elements are conjoined by “and/or”, a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or” as used herein.

As used herein, the term “pharmaceutically acceptable salts” refers to the relatively non-toxic, organic or inorganic salts of the active compounds, including inorganic or organic acid addition salts of the compound.

### EXAMPLES

CI-1040 is a white powder with a molecular weight (MW) of 478.67 and is soluble in DMSO, slightly soluble in ethanol and is practically insoluble in water. CI-1040 was dissolved in DMSO for the in vitro investigations. The final concentration of DMSO in culture media was 1%. For in vivo experiments CI-1040 was also dissolved in DMSO. Then, 50 μl DMSO containing Cl-1040 was mixed with 150 μl Cremophor EL and 800 μl PBS. CI-1040 was purchased from either Active Biochem or Selleckchem. Previously it was shown that two weeks oral dosing of CI-1040 at a daily dose of 200 mg/kg was not toxic to mice and reduced tumor growth. MEK was inhibited for nine hours in tumors of mice single dosed orally with CI-1040 at 150 mg/kg (Sebolt-Leopold et al., 1999).

### Example 1: CI-1040 Suppresses Replication of H1N1Pdm09 Virus In Vitro

Human lung epithelial cells (A549) were infected with either H1N1pdm09 A/Regensburg/06/2009 (MOI=0.005) or were used to determine the EC₅₀ and CC₅₀ values (FIG. 1 A, B). Human lung epithelial cells (A549) were infected with either H3N2 A/Victoria/03/1975 (MOI=0.05), H5N1 A/mallard/Bavaria/1/2006 (MOI=0.001), H7N7 A/FPV/Bratislava/79 (MOI=0.01) or H7N9 A/Anhui/1/2013 (MOI=0.005) (FIG. 1B); or with A/Mississippi/3/2001 wild-type or A/Mississippi/3/2001 carrying a specific mutation in the neuraminidase (H275Y) (MOI=0.005) (FIG. 1C). Samples were cultured at 37° C. After 30 min the inoculum was removed, cells were rinsed with PBS and supplemented with 500 μl IMDM or DMEM/BA-Medium (0.2% BA, 1 mM MgCl₂, 0.9 mM CaCl₂, 100 U/ml penicillin, 0.1 mg/ml streptomycin) and 0.6 μl TPCK-treated Trypsin (only for H1N1pdm09, H3N2, H7N9) in presence of different concentrations of CI-1040, 1 μM Tamiflu® (oseltamivir) or solvent control. After an incubation period of 18 h (H7N7) or 24 h (H1N1pdm09, H3N2, H5N1, H7N9) at 37° C. viral titers were determined by standard plaque assay. Viral titers are depicted as plaque forming units/ml (PFU/ml) and calculated to percent virus titer compared to solvent control, which was set to 100%. Data represent the means of one (H7N9) or two independent experiments with two (H7N9) or three (all other) biological replicates and two (H7N9) or three (all other) technical replicates. (FIG. 1A) Dose-dependent inhibition of A/Regensburg/06/2009 by CI-1040 (left panel) determination of the EC₅₀ for Cl-1040 (middle panel) and CC₅₀ (right panel). Note: a concentration of 100 μM didn't lead to a cytotoxic effect and therefore a sigmoidal curve could not be designed. Thus, the CC₅₀ value is an extrapolation by the Graphpad Prism software. (FIG. 1B) CI-1040 (10 μM) shows a broad antiviral range against different IAV. (FIG. 1C) Cl-1040 can inhibit a Tamiflu®-resistant IAV strain.

### Example 2: CI-1040 Blocks the Nuclear Cytoplasmic Transport of Viral RNP Complexes

Human lung epithelial cells (A549) were pre-treated with DMSO or 20 μM CI-1040 1 h before infection with IAV A/PR8/34 (H1N1) at a multiplicity of infection (MOI) of 5.30 min after the viral attachment at 4° C., attempts were rinsed with PBS and incubated 30 min at 37° C. with fresh medium for viral internalization. Cells were rinsed with PBS and supplemented 500 μl DMEM-Medium (6% BA, 1 mM MgCl2, 1 mM CaCl2, 100 U/ml penicillin, 0.1 mg/ml streptomycin) containing DMSO or CI-1040 were given to the cells. After the specific infection periods, samples were prepared for immunofluorescence staining of constituents of the viral RNP complexes, PB1 (second column) and NP (third column) and. Anti-NP (BioRad) and anti-PB 1 (Santa Cruz) were used to detect these viral proteins. Cell nuclei were stained with DAPI (first column).

The results in FIG. 2 show that CI-1040 blocks the export step of influenza A, H1N1, in human lung epithelial cells (A549).

### Example 3: CI-1040 is Effective in an In Vivo Model of Influenza Infection and has a Prolonged Therapy Window in Comparison to Tamiflu®

8 weeks old C57BL/6 mice (five per group) were infected with 1.5×10⁵ PFU (5×MLD50) of the influenza virus strain A/Regensburg/D6/2009, (RB1, H1N1pdm09) on day 0 (anesthetized with ketamine/rompun). Starting 8 h prior to infection mice were treated per os every 8 h with 125 mg/kg CI-1040 (CI-1040 dissolved in a combination of 50 μl DMSO/150 μl Cremophor/800 μl PBS) (see FIG. 3A, upper panel). Mice were sacrificed 24 h post infection and lungs were weighed, transferred into a Lysing Matrix D tube (MP Bio) and subsequently a 10-fold volume of the lung of BSS (buffered salt solution) was applied to the samples. Organs were shredded using the FastPrep FP 120 (Savant). To remove the cell debris the homogenates were centrifuged for 15 min at 2000 rpm and the supernatant collected. Determination of virus titer in the homogenates was performed using the AVICEL® plaque assay. The experiment was performed twice independently. FIG. 3A shows the efficacy of CI-1040 in the treatment of C57BL/6 mice infected with influenza virus H1N1pdm09.

In another set of experiments, 8 weeks old C57BL/6 mice (five per group) were infected with 1.5×10⁵ PFU (5×MLD50) of the influenza virus strain A/Regensburg/D6/2009, (RB1, H1N1pdm09). Starting either with the infection (FIG. 3B, left panel) or 24 h after infection (FIG. 3B, middle panel) or 48 h after infection (FIG. 3C, right panel) mice were treated with either 150 mg/kg/daily BID CI-1040 solved in 50 μl DMSO/150 μl Cremophor/800 μl PBS; or 4 mg/kg/daily BID Tamiflu® solved in H2O or solvent (placebo) control. All animals received a volume of 200 μl per os and were monitored twice daily and bodyweight was measured. Bodyweight prior to infection was set as 100% and increase/decrease of bodyweight was given in percentage. When an animal died, or had to be sacrificed according to the rules of the German animal protection law the bodyweight at this day was noted throughout the end of the experiment (day 14 p.i.). The overall clinical score was divided into four categories (score for breathing, surrounding, posture and grooming). For each category, an individual score from 0 to 3 is given resulting in an overall maximal score of 12. Nevertheless, mice were sacrificed when a total score of nine was reached or when a score of three was reached in two categories. Clinical score at the day of death was noted throughout the end of the experiment. The results in FIG. 3B show the efficacy of CI-1040 in the treatment of C57BL/6 mice infected with influenza virus H1N1pdm09 and proves the prolonged treatment window in comparison to oseltamivir (Tamiflu®).

### Example 4: Further In Vitro and In Vivo Studies

In Vitro Studies with the MEK-Inhibitor CI-1040 Against H5N1 Influenza Virus

MDCK II cells were infected with A/Mallard/Bavaria/01/2006 (MB 1, H5N1) at a MOI of 0.001. After 30 min. cells were treated with CI-1040 for 24 h.

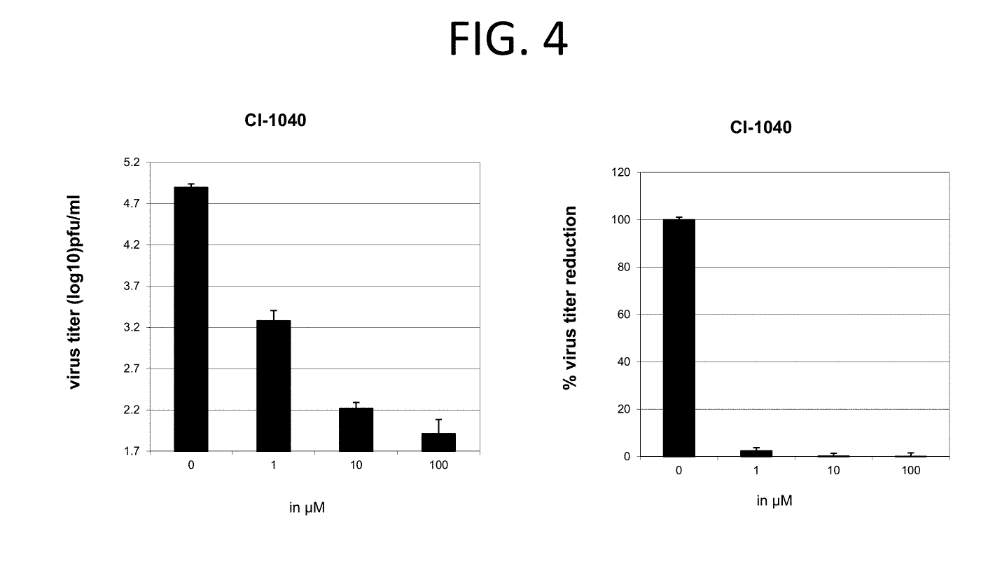

From FIG. 4 it can be seen that The MEK-Inhibitor CI-1040 is highly potent against different influenza virus strains in cell culture. CI-1040 is also potent against panH1N1 (RB1) and FPV.

Oral treatment of influenza virus infected mice with CI-1040 or PD-03250901

Eight hours prior infection BL/6 mice were treated (per os) with CI-1040, PD-03250901 (25 mg/kg) or solvent. At the time point of infection (A/Regensburg/D6/09, H1N1pdm09, RB1, 5-fold MLD50) mice were treated again, afterwards all eight hours (4 in total). Twenty-four hours after infection lung virus titer were detected. (n=5)

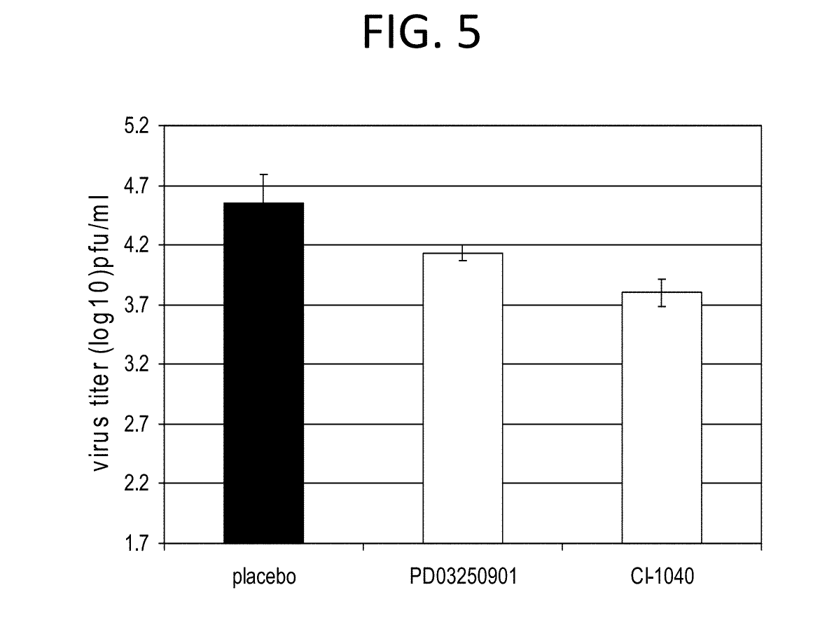

From FIG. 5 it can be seen that the MEK-Inhibitors CI-1040 and PD-03250901 are potent in reducing virus titer in the lung of H1N1pdm09 infected mice.

### REFERENCES

Aoki, F. Y., Boivin, G., Roberts, N., 2007. Influenza virus susceptibility and resistance to oseltamivir. Antivir Ther 12, 603-616.
Benn et al., J Virol 70, 4978-4985, 1996.
Bruder and Kovesdi, J Virol 71, 398-404, 1997.
Appiah, G. D., Blanton, L., D'Mello, T., Kniss, K., Smith, S., Mustaquim, D., Steffens, C., Dhara, R., Cohen, J., Chaves, S. S., Bresee, J., Wallis, T., Xu, X., Abd Elal, A. I., Gubareva, L., Wentworth, D. E., Katz, J., Jernigan, D., Brammer, L., Centers for Disease, C., Prevention, 2015. Influenza activity—United States, 2014-15 season and composition of the 2015-16 influenza vaccine. MMWR Morb Mortal Wkly Rep 64, 583-590.
Baranovich, T., Burnham, A. J., Marathe, B. M., Armstrong, J., Guan, Y., Shu, Y., Peiris, J. M., Webby, R. J., Webster, R. G., Govorkova, E. A., 2014. The neuraminidase inhibitor oseltamivir is effective against A/Anhui/1/2013 (H7N9) influenza virus in a mouse model of acute respiratory distress syndrome. J Infect Dis 209, 1343-1353.
Barrett, S. D., Bridges, A. J., Dudley, D. T., Saltiel, A. R., Fergus, J. H., Flamme, C. M., Delaney, A. M., Kaufman, M., LePage, S., Leopold, W. R., Przybranowski, S. A., Sebolt-Leopold, J., Van Becelaere, K., Doherty, A. M., Kennedy, R. M., Marston, D., Howard, W. A., Jr., Smith, Y., Warmus, J. S., Tecle, H., 2008. The discovery of the benzhydroxamate MEK inhibitors CI-1040 and PD 0325901. Bioorg Med Chem Lett 18, 6501-6504.
Bright, R. A., Shay, D. K., Shu, B., Cox, N. J., Klimov, A. I., 2006. Adamantane resistance among influenza A viruses isolated early during the 2005-2006 influenza season in the United States. Jama 295, 891-894.
Broberg, E., Snacken, R., Adlhoch, C., Beaute, J., Galinska, M., Pereyaslov, D., Brown, C., Penttinen, P., Region, W. H. O. E., the European Influenza Surveillance, N., 2015. Start of the 2014/15 influenza season in Europe: drifted influenza A(H3N2) viruses circulate as dominant subtype. Euro Surveill 20.
Cohen, Trends in Cell Biol 7, 353-361, 1997.
Droebner, K., Pleschka, S., Ludwig, S., Planz, O., 2011. Antiviral activity of the MEK-inhibitor U0126 against pandemic H1N1v and highly pathogenic avian influenza virus in vitro and in vivo. Antiviral Res 92, 195-203.
Fouchier, R. A., Schneeberger, P. M., Rozendaal, F. W., Broekman, J. M., Kemink, S. A., Munster, V., Kuiken, T., Rimmelzwaan, G. F., Schutten, M., Van Doornum, G. J., Koch, G., Bosman, A., Koopmans, M., Osterhaus, A. D., 2004. Avian influenza A virus (H7N7) associated with human conjunctivitis and a fatal case of acute respiratory distress syndrome. Proc Natl Acad Sci USA 101, 1356-1361.
Fremin and Meloche (2010), J. Hematol. Oncol. 11; 3:8.
Gong, J., Fang, H., Li, M., Liu, Y., Yang, K., Liu, Y., Xu, W., 2009. Potential targets and their relevant inhibitors in anti-influenza fields. Curr Med Chem 16, 3716-3739.
Gubareva et al., J Infect Dis 178, 1257-1262, 1998.
Jefferson, T., Jones, M., Doshi, P., Del Mar, C., 2009. Neuraminidase inhibitors for preventing and treating influenza in healthy adults: systematic review and meta-analysis. BMJ 339, b5106.
Jefferson, T., Jones, M. A., Doshi, P., Del Mar, C. B., Hama, R., Thompson, M. J., Spencer, E. A., Onakpoya, I., Mahtani, K. R., Nunan, D., Howick, J., Heneghan, C. J., 2014. Neuraminidase inhibitors for preventing and treating influenza in healthy adults and children. The Cochrane database of systematic reviews 4, CD008965.
Jernigan, D. B., Cox, N. J., 2015. H7N9: preparing for the unexpected in influenza. Annu Rev Med 66, 361-371.
Kiso, M., Mitamura, K., Sakai-Tagawa, Y., Shiraishi, K., Kawakami, C., Kimura, K., Hayden, F. G., Sugaya, N., Kawaoka, Y., 2004. Resistant influenza A viruses in children treated with oseltamivir: descriptive study. Lancet 364, 759-765.
Lackenby, A., Thompson, C. I., Democratis, J., 2008. The potential impact of neuraminidase inhibitor resistant influenza. Curr Opin Infect Dis 21, 626-638.
Leneva, I. A., Roberts, N., Govorkova, E. A., Goloubeva, O. G., Webster, R. G., 2000. The neuraminidase inhibitor GS4104 (oseltamivir phosphate) is efficacious against A/Hong Kong/156/97 (H5N1) and A/Hong Kong/1074/99 (H9N2) influenza viruses. Antiviral Res 48, 101-115.
Lorusso, P. M., Adjei, A. A., Varterasian, M., Gadgeel, S., Reid, J., Mitchell, D. Y., Hanson, L., DeLuca, P., Bruzek, L., Piens, J., Asbury, P., Van Becelaere, K., Herrera, R., Sebolt-Leopold, J., Meyer, M. B., 2005. Phase I and pharmacodynamic study of the oral MEK inhibitor CI-1040 in patients with advanced malignancies. J Clin Oncol 23, 5281-5293.
Ludwig, S., 2009. Targeting cell signalling pathways to fight the flu: towards a paradigm change in anti-influenza therapy. J Antimicrob Chemother 64, 1-4.
Ludwig, S., 2011. Disruption of virus-host cell interactions and cell signaling pathways as an anti-viral approach against influenza virus infections. Biol Chem 392, 837-847.
Ludwig, S., Planz, O., Pleschka, S., Wolff, T., 2003. Influenza-virus-induced signaling cascades: targets for antiviral therapy? Trends Mol Med 9, 46-52.
Ludwig, S., Wolff, T., Ehrhardt, C., Wurzer, W. J., Reinhardt, J., Planz, O., Pleschka, S., 2004. MEK inhibition impairs influenza B virus propagation without emergence of resistant variants. FEBS Lett 561, 37-43.
Mackey, T. K., Liang, B. A., 2012. Lessons from SARS and H1N1/A: employing a WHO-WTO forum to promote optimal economic-public health pandemic response. J Public Health Policy 33, 119-130.
Marjuki, H., Yen, H. L., Franks, J., Webster, R. G., Pleschka, S., Hoffmann, E., 2007. Higher polymerase activity of a human influenza virus enhances activation of the hemagglutinin-induced Raf/MEK/ERK signal cascade. Virol J 4, 134.
Mazur, I., Wurzer, W. J., Ehrhardt, C., Pleschka, S., Puthavathana, P., Silberzahn, T., Wolff, T., Planz, O., Ludwig, S., 2007. Acetylsalicylic acid (ASA) blocks influenza virus propagation via its NF-kappaB-inhibiting activity. Cell Microbiol 9, 1683-1694.
Monto, A. S., Black, S., Plotkin, S. A., Orenstein, W. A., 2011. Response to the 2009 pandemic: effect on influenza control in wealthy and poor countries. Vaccine 29, 6427-6431.
Morens, D. M., Taubenberger, J. K., Fauci, A. S., 2013. H7N9 avian influenza A virus and the perpetual challenge of potential human pandemicity. MBio 4.
Morrison, J., Josset, L., Tchitchek, N., Chang, J., Belser, J. A., Swayne, D. E., Pantin-Jackwood, M. J., Tumpey, T. M., Katze, M. G., 2014. H7N9 and other pathogenic avian influenza viruses elicit a three-pronged transcriptomic signature that is reminiscent of 1918 influenza virus and is associated with lethal outcome in mice. J Virol 88, 10556-10568.
Moss, R. B., Davey, R. T., Steigbigel, R. T., Fang, F., 2010. Targeting pandemic influenza: a primer on influenza antivirals and drug resistance. J Antimicrob Chemother 65, 1086-1093.
Olschlager, V., Pleschka, S., Fischer, T., Rziha, H. J., Wurzer, W., Stitz, L., Rapp, U. R., Ludwig, S., Planz, O., 2004. Lung-specific expression of active Raf kinase results in increased mortality of influenza A virus-infected mice. Oncogene 23, 6639-6646.
Osterholm, M. T., Kelley, N. S., Sommer, A., Belongia, E. A., 2012. Efficacy and effectiveness of influenza vaccines: a systematic review and meta-analysis. Lancet Infect Dis 12, 36-44.
Planz, O., 2013. Development of cellular signaling pathway inhibitors as new antivirals against influenza. Antiviral Res 98, 457-468.
Planz, O., Pleschka, S., Ludwig, S., 2001. MEK-specific inhibitor U0126 blocks spread of Borna disease virus in cultured cells. J Virol 75, 4871-4877.
Pleschka, S., Wolff, T., Ehrhardt, C., Hobom, G., Planz, O., Rapp, U. R., Ludwig, S., 2001. Influenza virus propagation is impaired by inhibition of the Raf/MEK/ERK signalling cascade. Nat Cell Biol 3, 301-305.
Popik and Pitha, Virology 252, 210-217, 1998.
Robertson, J. S., Inglis, S. C., 2011. Prospects for controlling future pandemics of influenza. Virus Res 162, 39-46.
Robinson and Cobb, Curr. Opin. Cell Biol 9, 180-186, 1997.
Rodems and Spector, J Virol 72, 9173-9180, 1998.
Scholtissek and Miiller, Arch Virol 119, 111-118, 1991.
Sebolt-Leopold, J. S., Dudley, D. T., Herrera, R., Van Becelaere, K., Wiland, A., Gowan, R. C., Tecle, H., Barrett, S. D., Bridges, A., Przybranowski, S., Leopold, W. R., Saltiel, A. R., 1999. Blockade of the MAP kinase pathway suppresses growth of colon tumors in vivo. Nat Med 5, 810-816.
Spence, J. K., Bhattachar, S. N., Wesley, J. A., Martin, P. J., Babu, S. R., 2005. Increased dissolution rate and bioavailability through comicronization with microcrystalline cellulose. Pharm Dev Technol 10, 451-460.
Thorlund, K., Awad, T., Boivin, G., Thabane, L., 2011. Systematic review of influenza resistance to the neuraminidase inhibitors. BMC Infect Dis 11, 134.
Treisman, Curr. Opin. Cell Biol 8, 205-215, 1996.
Yen, H. L., Monto, A. S., Webster, R. G., Govorkova, E. A., 2005. Virulence may determine the necessary duration and dosage of oseltamivir treatment for highly pathogenic A/Vietnam/1203/04 influenza virus in mice. J Infect Dis 192, 665-672.
York, I., Donis, R. O., 2013. The 2009 pandemic influenza virus: where did it come from, where is it now, and where is it going? Curr Top Microbiol Immunol 370, 241-257.

1. A method for treating a subject having a viral infection, comprising administering to the subject the MEK inhibitor CI-1040 or a pharmaceutically acceptable salt or derivative thereof, wherein administering treats a viral infection in the subject.

2. The method according to claim 1, wherein the subject has been symptomatic for the viral infection for at least 24 hours when treating is started.

3. The method according to claim 1, wherein the subject has been symptomatic for the viral infection for at least 36 hours when treating is started.

4. The method according to claim 1, wherein the subject has been symptomatic for the viral infection for at least 48 hours when treating is started.

5. The method according to claim 1, wherein the treating is started at least 24 hours and within 48 hours post onset of disease.

6. The method according to claim 1, wherein the viral infection is caused by a negative RNA strand virus.

7. The method according to claim 6, wherein the virus is influenza virus.

8. The method according to claim 7, wherein the virus is influenza A virus or influenza B virus.

9. The method according to claim 8, wherein the influenza A virus is selected from the group consisting according to H1N1, H2N2, H3N2, H5N6, H5N8, H6N1, H7N2, H7N7, H7N9, H9N2, H10N7, N10N8 or H5N1.

10. The method according to claim 7, wherein the Influenza B virus is selected from the group consisting of IBV-type Yamagata or Victoria.

11. The method according to claim 1, wherein the virus is resistant to standard antiviral treatment.

12. The method according to claim 11, wherein the standard antiviral treatment is administration of oseltamivir, zanamivir, peramivir, amantadine, rimantadine, favipiravir, baloxavir marboxil and/or pimodivir.

13. The method according to claim 9, wherein the virus is H1N1 virus a H275Y mutant.

14. The method according to claim 1, wherein the subject is human.

15. The method according to claim 1, wherein the subject has been treated with a standard antiviral prior to administration of CI-1040
